(19) United States
(12) Patent Application Publication
(10) Pub. No.: US 2025/0289858 A1
(43) Pub. Date: Sep. 18, 2025

(54) BAG3 AND PROTEIN QUALITY CONTROL IN THE BRAIN

(71) Applicant: TEMPLE UNIVERSITY OF THE COMMONWEALTH SYSTEM OF HIGHER EDUCATION, Philadelphia, PA (US)

(72) Inventors: Arthur M. FELDMAN, Wynnewood, PA (US); Valerie MYERS, Arlington, VA (US)

(73) Assignee: TEMPLE UNIVERSITY OF THE COMMONWEALTH SYSTEM OF HIGHER EDUCATION, Philadelphia, PA (US)

(21) Appl. No.: 18/861,495

(22) PCT Filed: May 2, 2023
(86) PCT No.: PCT/US2023/020659

§ 371 Date: Oct. 29, 2024

Related U.S. Application Data
(60) Provisional application No. 63/337,914, filed on May 3, 2022.

(51) Int. Cl. C07K 14/47; A61K 38/00

(52) U.S. Cl. CPC C07K 14/4702; A61K 38/00

(57) ABSTRACT

BAG3 protein is used in the treatment of, for example, is Parkinson's disease, Alzheimer's disease, Amyotrophic Lateral Sclerosis, Huntington disease, Lewy body disease, vascular dementia, mixed dementia and Traumatic Brain Injury, for example, complicated by Chronic Traumatic Encephalopathy (CTE). Therapeutically effective BAG3 compositions, BAG3 uses and BAG3 methods of treatment are described.

### RELATED APPLICATIONS

This application claims priority to U.S. Provisional Patent Application No. 63/337,914, filed May 3, 2022. The entire content of the foregoing application is incorporated herein by reference, including all text, tables, sequence listings and drawings.

### GOVERNMENT SUPPORT

This invention was made with government support under Grant No. HL91799 awarded by the National Institutes of Health. The government has certain rights in the invention.

### FIELD OF THE INVENTION

Embodiments of the invention are directed to BAG3 protein quality control in diseases or disorders of the brain, central nervous system, neurological, including traumatic injuries.

### BACKGROUND

BAG3 plays a critical role in the heart by regulating protein quality control through enhanced autophagy and decreased apoptosis. Most recently we identified a group of functional mutations in BAG3 that were found only in individuals of African ancestry which resulted in a nearly 2-fold increase in the risk of either death or worsening heart failure requiring hospitalization.

### SUMMARY

Embodiments of the invention are directed to BAG3 protein quality control in diseases or disorders of the brain, central nervous system, neurological, including traumatic injuries. Examples, include, but not limited to Parkinson's Disease, Alzheimer's Disease and Traumatic Brain Injury complicated by Chronic Traumatic Encephalopathy.

Embodiments of the invention may be practiced without the theoretical aspects presented. Moreover, the theoretical aspects are presented with the understanding that Applicants do not seek to be bound by the theory presented.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes disclosed herein, which in some embodiments relate to mammalian nucleic acid and amino acid sequences are intended to encompass homologous and/or orthologous genes and gene products from other animals including, but not limited to other mammals, fish, amphibians, reptiles, and birds. In preferred embodiments, the genes or nucleic acid sequences are human.

### Definitions

The terminology used herein is for the purpose of describing particular embodiments only and is not intended to be limiting of the invention. As used herein, the singular forms “a,” “an” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

As used herein “BAG3,” “BAG3 molecules,” “BCL2-associated athanogene 3 (BAG3) genes,” “BCL2-associated athanogene 3 (BAG3) molecules” are inclusive of all family members, mutants, cDNA sequences, alleles, fragments, species, coding and noncoding sequences, sense and antisense polynucleotide strands, etc. (HGNC (939) Entrez Gene (9531) Ensembl (ENSG00000151929) OMIM (603883) UniProtKB (095817)). Similarly, “BAG3,” “BAG3 molecules,” “BCL2-associated athanogene 3 (BAG3) molecules” also refer to BAG3 polypeptides or active fragment thereof, proteins, variants, derivatives etc. The term “molecule,” thus encompasses both the nucleic acid sequences and amino acid sequences of BAG3.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

“Optional” or “optionally” means that the subsequently described circumstance may or may not occur, such that the description includes instances where the circumstance occurs and instances where it does not.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

A “recombinant viral vector” refers to a viral vector comprising one or more heterologous gene products or sequences. Since many viral vectors exhibit size-constraints associated with packaging, the heterologous gene products or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying gene products necessary for replication and/or encapsidation). Modified viral vectors in which a polynucleotide to be delivered is carried on the outside of the viral particle have also been described (see, e.g., Curiel, D T, et al., PNAS 88: 8850-8854, 1991).

By the term “modulate,” it is meant that any of the mentioned activities of the compounds embodied herein, are, e.g., increased, enhanced, increased, agonized (acts as an agonist), promoted, decreased, reduced, suppressed blocked, or antagonized (acts as an antagonist). Modulation can increase activity more than 1-fold, 2-fold, 3-fold, 5-fold, 10-fold, 100-fold, etc., over baseline values. Modulation can also decrease its activity below baseline values.

As used herein, the term “agent” is meant to encompass any molecule, chemical entity, composition, drug, therapeutic agent, chemotherapeutic agent, or biological agent capable of preventing, ameliorating, or treating a disease or other medical condition. The term includes small molecule compounds, antisense reagents, siRNA reagents, antibodies, enzymes, peptides organic or inorganic molecules, natural or synthetic compounds and the like. An agent can be assayed in accordance with the methods of the invention at any stage during clinical trials, during pre-trial testing, or following FDA-approval.

As defined herein, a “therapeutically effective” amount of a compound or agent (i.e., an effective dosage) means an amount sufficient to produce a therapeutically (e.g., clinically) desirable result. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments.

“Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. Accordingly, “treating” or “treatment” of a state, disorder or condition includes; (1) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition; (2) inhibiting the state, disorder or condition, i.e., arresting, reducing or delaying the development of the disease or a relapse thereof (in case of maintenance treatment) or at least one clinical or subclinical symptom thereof; or (3) relieving the disease, i.e., causing regression of the state, disorder or condition or at least one of its clinical or subclinical symptoms. The benefit to an individual to be treated is either statistically significant or at least perceptible to the patient or to the physician.

### DETAILED DESCRIPTION

While tau and alpha-synuclein are known to play a role in disease, the possibility that BAG3 plays a critical role in clearing them from the brain has not been developed prior to this invention. Furthermore, the concept that individuals with loss of function variants in BAG3 would be at high risk for diseases of the brain that are associated with abnormalities in protein quality control has also not been previously shown. Lastly, the use of BAG3 as a therapeutic in Parkinson's Disease, Alzheimer's Disease and CTE has not been previously addressed.

In certain embodiments, an agent modulates expression or amount of BCL2-associated athanogene 3 (BAG3) molecules, proteins or peptides thereof in a target cell or tissue, as compared to a normal control, comprising an expression vector expressing a BAG3 protein or active fragments thereof, oligonucleotides or combinations thereof.

In certain embodiments, an agent induces expression of BAG3. In certain embodiments the agent comprises proteins or peptides thereof, peptidomimetics, small molecules, organic or inorganic compounds, synthetic or natural compounds.

Bcl-2 associated anthanogene-3 (BAG3), also known as BCL2-Associated Athanogene 3; MFM6; Bcl-2-Binding Protein Bis; CAIR-1; Docking Protein CAIR-1; BAG Family Molecular Chaperone Regulator 3; BAG-3; BCL2-Binding Athanogene 3; or BIS, is a cytoprotective polypeptide that competes with Hip-1 for binding to HSP 70. The NCBI reference amino acid sequence for BAG3 can be found at Genbank under accession number NP_004272.2; Public GI:14043024. The NCBI reference nucleic acid sequence for BAG3 can be found at Genbank under accession number NM_004281.3 GI:62530382. Other BAG3 amino acid sequences include, for example, without limitation, 095817.3 GI:12643665; EAW49383.1 GI:119569768; EAW49382.1 GI:119569767; and CAE55998.1 GI:38502170. The BAG3 polypeptide of the invention can be a variant of a polypeptide described herein, provided it retains functionality.

TABLE 1
| Exemplary gene promoters driving expression in the
Central Nervous System (CNS)¹

| | Cortical | Neuropeptide/ | Widespread | |
|---|---|---|---|---|
| | inter- | neuro- | brain | Regionally |
| Cortical layer | neuron | modulator | expression | enriched |
| A930038C07Rik | Calb2 | Adcyap1 | Dcx | Camk2a |
| Chrna2 | Cck | Agrp | Eno2 | Cart |
| Chrnb4 | Cort | Avp | Gfap | Cdhr1 |
| Ctgf | Crh | Chat | Nefh | Cnnm2 |
| Cux2 | Dlx1 | Dbh | Nes | Cyp39al |
| Cux2 | Dlx5 | Gal | Otof | Dbx1 |
| Cux2 | Erbb4 | Gnrh1 | Slc17a8 | Drd1a |
| Efr3a | Gad2 | Hdc | Snap25 | Drd2 |
| Etv1 | Nkx2-1 | Ins2 | Syn1 | Drd3 |
| Gng7 | Nos1 | Kiss1 | Vamp2 | Emx1 |
| Gpr26 | Pvalb | Oxt | | Esr1 |
| Grp | Pvalb | Penk | | Gabra6 |
| Htr2a | Slc32al | Pmch | | Gabrr3 |
| Nr5a1 | Sst | Pomc | | Grik4 |
| Ntsr1 | Tac1 | Slc6a3 | | Grm2 |
| Nxph4 | Tac2 | Slc6a4 | | Kcnc2 |
| Otof | Vip | Slc18a2 | | Lepr |
| Rasgrf2 | | Th | | Lhx6 |
| Rbp4 | | Ucn3 | | Lypd6 |
| Rorb | | | | Mybpc1 |
| Scnnla | | | | Nefl |
| Sim 1 | | | | Ntrk1 |
| Six3 | | | | Oxtr |
| Syt6 | | | | Pcdh9 |
| Trib2 | | | | Pcp2 |
| Wfs1 | | | | Pdzklip1 |
| | | | | Plxnd1 |
| | | | | Pnmt |
| | | | | Ppp1r17 |
| | | | | Prkcd |
| | | | | Satb2 |
| | | | | Sim 1 |
| | | | | Slc6a5 |
| | | | | Slc17a6 |
| | | | | Slc17a7 |
| | | | | Syn1 |
| | | | | Svt17 |
| | | | | Tlx3 |
| | | | | Vipr2 |
| | | | | Wnt3a |

¹Table adapted from Harris J. et al. Anatomical characterization of Cre driver mice for neural circuit mapping and manipulation. Front Neural Circuits. 2014; 8. doi: 10.3389/fncir.2014.00076

### EXAMPLES

Abnormalities in protein quality control are thought to be important in the pathobiology of Alzheimer's Disease, Chronic Traumatic Encephalopathy complicating traumatic brain injury and Parkinson's Disease. It was hypothesized as to whether abnormalities in BAG3 might contribute to the pathobiology of these central nervous system diseases and potentially serve as a therapeutic target. In addition, whether the development of these central nervous system diseases might be more common in individuals harboring mutations in BAG3. It is generally assumed that BAG3 is found predominantly in the heart and the skeletal muscle and little attention has been paid to its role in the brain.

Results from experiments supported the hypothesis that BAG3 plays a role in neurodegenerative diseases of the brain and can serve as an important therapeutic target: 1) BAG3 levels are expressed at high levels in the brain of mice in fact, by contrast with earlier reports, levels in the brain are comparable to those found in the heart; 2) BAG3 couples tightly with both tau and alpha-sinuclein, peptides that play an important role in Alzheimer's/CTE and Parkinson's Disease respectively; and 3) Common (>1%) loss of function genetic variants found in individuals of African ancestry fail to bind to these disease causing peptides.

What is claimed:

1. A method of treating a neurodegenerative disease of the brain or central nervous system comprising administering to a subject having or at risk of having a neurodegenerative disease of the brain or central nervous system a Bcl2-associated anthanogene 3 (BAG3) polynucleotide, polypeptide, or active fragments thereof to provide BAG3 polypeptide or active fragment thereof expression to the brain or central nervous system thereby treating or reducing the risk of the neurodegenerative disease of the brain or central nervous system.

2. The method of claim 1, wherein the neurodegenerative disease of the brain or central nervous system is Parkinson's disease, Alzheimer's disease, Amyotrophic Lateral Sclerosis, Huntington disease, Lewy body disease, vascular dementia or mixed dementia.

3. A method of treating traumatic brain injury comprising administering to a subject having or at risk of having a traumatic brain injury a Bcl2-associated anthanogene 3 (BAG3) polynucleotide, polypeptide or active fragment thereof to provide BAG3 polypeptide or active fragments thereof expression to the brain or central nervous system thereby treating or reducing the risk of having traumatic brain injury.

4. The method of claim 3, wherein the traumatic brain injury is traumatic brain injury complicated by Chronic Traumatic Encephalopathy (CTE).

5. The method of any of claims 1-3, wherein the expression or amount of BAG3 polynucleotide, polypeptide or active fragment thereof is increased in a brain or central nervous system cell or tissue, as compared to a normal control.

6. The method of any of claims 1-3, wherein the BAG3 polynucleotide comprises an expression vector for expression of the BAG3 polypeptide or active fragment thereof.

7. The method of claim 6, wherein the expression vector further comprises an expression control element.

8. The method of claim 7, wherein the expression control element comprises a promoter or enhancer.

9. The method of claim 8, wherein the promoter or enhancer comprises an inducible promoter or enhancer, a constitutive promoter or enhancer, bicistronic promoter or enhancer, or tissue specific promoter or enhancer.

10. The method of claim 7 or 8, wherein the expression control element, promoter or enhancer is active in the brain or central nervous system.

11. The method of claim 10, wherein the expression control element, promoter or enhancer active in the brain or central nervous system is selected from an expression control element, promoter or enhancer set forth in Table 1.

12. The method of claim 7, wherein the expression control element comprises a cytomegalovirus (CMV) promoter.

13. The method of claim 6, wherein the expression vector crosses the blood-brain barrier.

14. The method of any of claims 1-3, wherein the BAG3 polynucleotide, polypeptide or active fragment thereof is transmittable across the blood brain barrier or directly administered to the brain or central nervous system.

15. The method of claim 6, wherein the expression vector comprises a viral vector, plasmid, or a yeast vector.

16. The method of claim 15, wherein the viral vector comprises an adeno-associated virus (AAV) vector, an adenoviral vector, a lentiviral vector, a coxsackie viral vector, a cytomegalovirus vector, retroviral vector or an Epstein Barr virus vector.

17. The method of claim 16, wherein the AAV vector comprises a recombinant AAV (rAAV) particle comprising an AAV capsid protein and a vector genome comprising the polynucleotide encoding the BAG3 polypeptide or active fragment thereof.

18. The method of claim 17, wherein the vector genome further comprises one or more AAV inverted terminal repeat sequences (ITRs), an intron, a stop codon, or a poly-A sequence.

19. The method of claim 17 or 18, wherein the AAV capsid protein or AAV ITR(s) comprise:
(i) a capsid sequence having 70% or more identity to an AAV1, AAV2, AAV3, AAV-3B AAV4, AAV5, AAV6, AAV7, AAV8, AAV9, AAV10, Rh10, Rh74 or AAV-F VP1 capsid sequence, and/or
(ii) an ITR having 70% or more identity to an AAV1, AAV2, AAV3, AAV4, AAV5, AAV6, AAV7, AAV8, AAV9, AAV10, AAV11, Rh10, Rh74 or AAV-2i8 AAV ITR sequence.

20. The method of claim 18 or 19, wherein the AAV ITR(s) flanks the 5′or 3′ terminus of said polynucleotide encoding the BAG3 polypeptide or active fragment thereof.

21. The method of any one of claims 1-20, wherein the BAG3 polynucleotide, BAG3 polypeptide, active fragment thereof, expression vector or viral vector comprising a BAG3 polynucleotide is comprised in a pharmaceutical composition.

22. The method of any one of claims 1-20, wherein the BAG3 polynucleotide, BAG3 polypeptide, active fragment thereof, expression vector or viral vector comprising a BAG3 polynucleotide is comprised in microvesicles, nanovesicles or nanoparticles.

23. The method of any of claims 6-20, wherein the expression vector or viral vector targets the brain cortex.

24. A composition comprising a Bcl2-associated anthanogene 3 (BAG3) polynucleotide, polypeptide and/or agent which induce BAG3.

25. The composition of claim 24, wherein the agent modulates expression or amount of BCL2-associated athanogene 3 (BAG3) molecules, proteins or peptides thereof in a target cell or tissue, as compared to a normal control.

26. The composition of claim 24, wherein the agent comprises an expression vector expressing a BAG3 protein or active fragments thereof, oligonucleotides or combinations thereof.

27. The composition of claim 26, wherein the expression vector further comprises a promoter, the promoter comprising an inducible promoter, a constitutive promoter, bicistronic promoter or tissue specific promoter.

28. The composition of claim 26, wherein the expression vector comprises a viral vector, plasmid, or a yeast vector.

29. The composition of claim 26, wherein the vector crosses the blood-brain barrier.

30. The composition of claim 24, wherein the BAG3 or agent that induces the BAG3 is transmittable across the blood brain barrier or directly administered.

31. The composition of claim 24, wherein the agent comprises proteins or peptides thereof, peptidomimetics, small molecules, organic or inorganic compounds, synthetic or natural compounds.
